(19) United States
(12) Patent Application Publication
(10) Pub. No.: US 2022/0096674 A1
(43) Pub. Date: Mar. 31, 2022

(54) STERILIZATION CASE

(71) Applicant: Aevoe Inc., Taipei City (TW)

(72) Inventors: Shan Pei Hu, Taipei (TW); Shau Dong Jen, Taipei (TW); Fu Chin Shen, Taipei (TW); Huai Shun Chiang, Taipei (TW); Chih Kang Chen, Taipei (TW)

(73) Assignee: Aevoe Inc., Taipei City (TW)

(21) Appl. No.: 17/180,852

(22) Filed: Feb. 21, 2021

(30) Foreign Application Priority Data
Sep. 30, 2020 (TW) 109134303

(51) Int. Cl. A61L 2/10; A61L 2/26

(52) U.S. Cl. CPC A61L 2/10; A61L 2/26; A61L 2202/14; A61L 2202/121; A61L 2202/11; A61L 2202/122

(57) ABSTRACT

A sterilization case includes a case body, a transparent plate, and at least one ultraviolet light source. The case body is adapted to contain an object. The transparent plate is disposed in the case body and has a supporting surface. When the transparent plate supports the object, a surface of the object leans against the supporting surface. The ultraviolet light source is disposed in the case body and adapted to provide ultraviolet light, and the ultraviolet light passes through an interior of the transparent plate and is transmitted toward the supporting surface to irradiate the surface of the object.

### CROSS-REFERENCE TO RELATED APPLICATION

This application claims the priority benefit of Taiwanese application serial no. 109134303, filed on Sep. 30, 2020. The entirety of the above-mentioned patent application is hereby incorporated by reference herein and made a part of this specification.

### BACKGROUND

### Technical Field

The disclosure relates to a sterilization device; particularly, the disclosure relates to a sterilization case.

### Description of Related Art

With increasing awareness of environmental sanitation, various sterilization devices are commonly used in various public places and gradually popularized in families. In terms of an ultraviolet light sterilization device, when ultraviolet light is used to irradiate objects to be sterilized, it is required that the operator will not be irradiated by the ultraviolet light. For example, an object may be placed in a case body, and ultraviolet light for sterilization may be provided by an ultraviolet light source in the case body. However, when a surface of the object leans against a bearing surface in the case body, it is difficult for this surface of the object to be irradiated by the ultraviolet light, which leads to incomplete sterilization.

### SUMMARY

The disclosure provides a sterilization case that comprehensively sterilizes an object contained therein.

The sterilization case of the disclosure includes a case body, a transparent plate, and at least one ultraviolet light source. The case body is adapted to hold an object. The transparent plate is disposed in the case body and has a supporting surface. When the transparent plate supports the object, a surface of the object leans against the supporting surface. The ultraviolet light source is disposed in the case body and is adapted to provide ultraviolet light. The ultraviolet light passes through an interior of the transparent plate and is transmitted toward the supporting surface to irradiate the surface of the object.

In an embodiment of the disclosure, a material of the transparent plate includes cyclic block copolymer.

In an embodiment of the disclosure, the sterilization case includes a reflective layer. The reflective layer is disposed on a bottom surface of the case body. The transparent plate is disposed on the reflective layer. The reflective layer is adapted to reflect the ultraviolet light toward the supporting surface.

In an embodiment of the disclosure, a material of the reflective layer includes Teflon.

In an embodiment of the disclosure, the sterilization case includes at least one reflective layer. The at least one reflective layer is disposed on at least one inner surface of the case body and is adapted to reflect the ultraviolet light toward the transparent plate or the object.

In an embodiment of the disclosure, a material of the reflective layer includes metal.

In an embodiment of the disclosure, the at least one ultraviolet light source comprises a plurality of ultraviolet light sources. A height of a part of the plurality of ultraviolet light sources relative to a bottom surface of the case body is different from a height of another part of the plurality of ultraviolet light sources relative to the bottom surface of the case body.

In an embodiment of the disclosure, the transparent plate has a photosensitive material. The photosensitive material is adapted to display a visible text or graphic after being irradiated by the ultraviolet light.

In an embodiment of the disclosure, the case body is adapted to be folded into a folded state and adapted to be unfolded into an upright state.

In an embodiment of the disclosure, the sterilization case includes a magnetic element and a magnetic sensing element. The case body includes a case part and a cover part connected to each other. The transparent plate and the at least one ultraviolet light source are disposed in the case part. The magnetic element is disposed in one of the case part and the cover part, and the magnetic sensing element is disposed in the other one of the case part and the cover part. When the cover part covers the case part, the magnetic sensing element senses that the magnetic sensing element and the magnetic element are close to each other and accordingly triggers the at least one ultraviolet light source to be turned on. When the cover part is unfolded relative to the case part, the magnetic sensing element senses that the magnetic sensing element and the magnetic element are away from each other and accordingly triggers the at least one ultraviolet light source to be turned off.

In an embodiment of the disclosure, the aforementioned sterilization case includes a light sensing element. The case body includes a case part and a cover part connected to each other. The transparent plate, the at least one ultraviolet light source, and the light sensing element are disposed in the case part. When the cover part is unfolded relative to the case part, the light sensing element senses external ambient light and accordingly triggers the at least one ultraviolet light source to be turned off.

In an embodiment of the disclosure, the sterilization case includes a wire fixing belt. The wire fixing belt has a first segment and a second segment. The first segment is connected to an outer side of the case body. The second segment is adapted to be reversely folded relative to the first segment and attached to the first segment.

In an embodiment of the disclosure, the sterilization case includes a status indicator. The status indicator is disposed on an outer side of the case body. When the at least one ultraviolet light source is turned on, the status indicator emits a first color light. When the at least one ultraviolet light source is turned off, the status indicator emits a second color light different from the first color light.

In an embodiment of the disclosure, the transparent plate has two finger receiving grooves. The two finger receiving grooves are respectively located on two opposite ends of the supporting surface.

Based on the foregoing, in the sterilization case in the disclosure, the transparent plate that supports the object to be sterilized is disposed in the case body, so that the ultraviolet light emitted by the ultraviolet light sources passes through the transparent plate and irradiates the surface of the object that leans against the transparent plate, achieving comprehensive sterilization.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a perspective view of a sterilization case according to an embodiment of the disclosure.

FIG. 2 shows a cover part of FIG. 1 expanding relative to a case part.

FIG. 3 shows the transparent plate of FIG. 2 supporting an object.

FIG. 4 is a schematic partial cross-sectional view of the sterilization case of FIG. 3.

FIG. 5 is a perspective view of the sterilization case of FIG. 3 from another view angle.

FIG. 6 is a perspective view of the sterilization case of FIG. 1 from another view angle.

FIG. 7 shows the sterilization case of FIG. 1 folded into a folded state.

### DESCRIPTION OF THE EMBODIMENTS

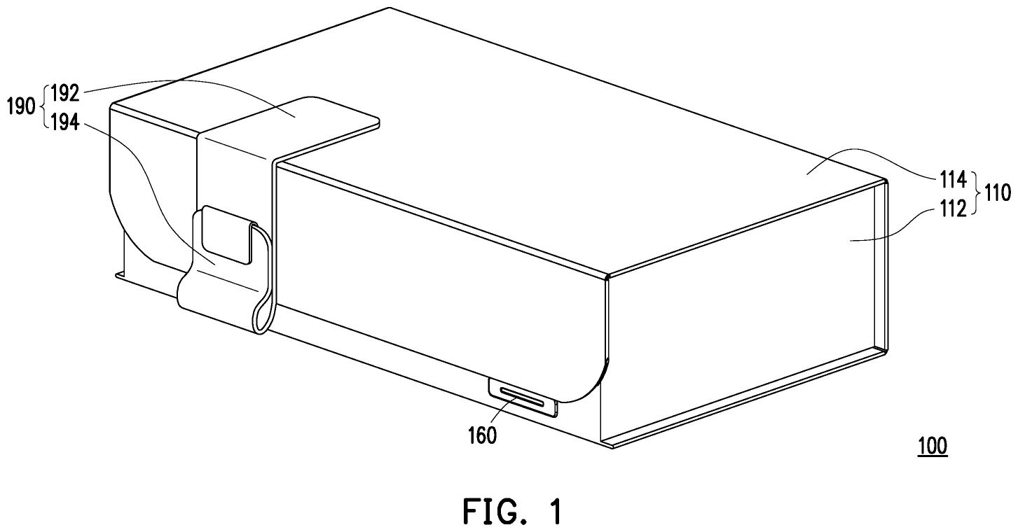

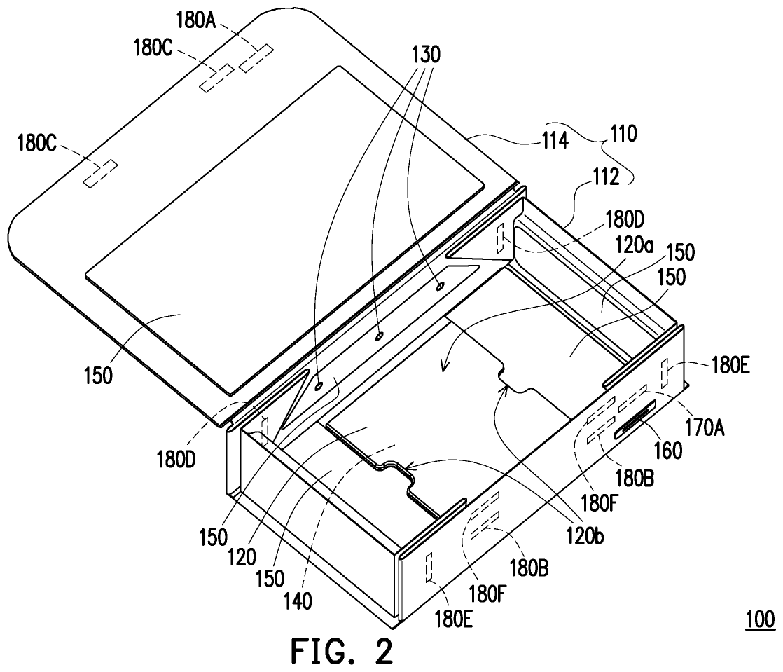

FIG. 1 is a perspective view of a sterilization case according to an embodiment of the disclosure. FIG. 2 shows a cover part of FIG. 1 expanding relative to a case part. With reference to FIG. 1 and FIG. 2, a sterilization case 100 of the embodiment includes a case body 110, a transparent plate 120, and at least one ultraviolet light source 130 (shown as plural). The case body 110 includes a case part 112 and a cover part 114 connected to each other. The transparent plate 120 and the ultraviolet light source 130 are disposed in the case part 112. The cover part 114 may cover the case part 112 as shown in FIG. 1, and may be expanded relative to the case part 112 as shown in FIG. 2.

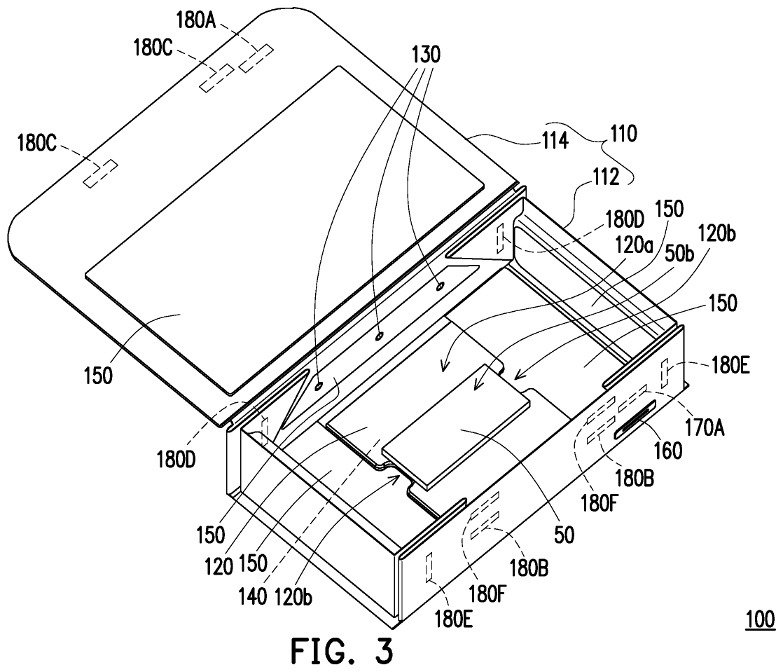

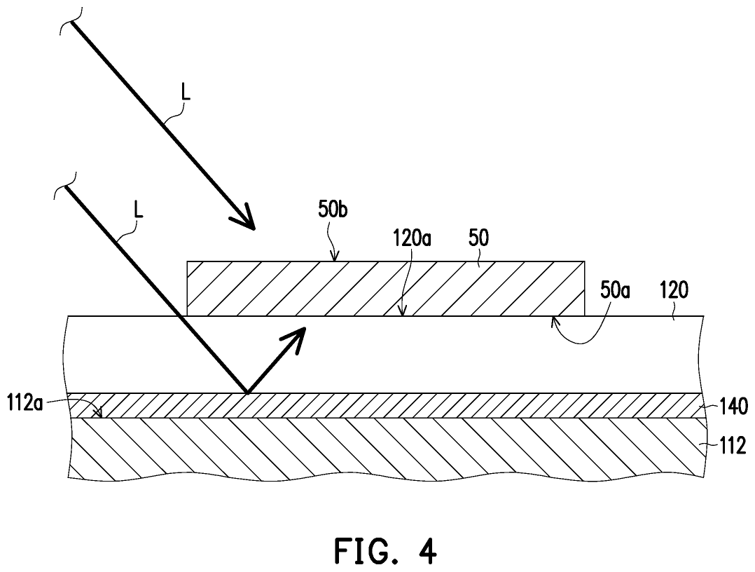

FIG. 3 shows the transparent plate of FIG. 2 supporting an object. FIG. 4 is a schematic partial cross-sectional view of the sterilization case of FIG. 3. The case part 112 of the case body 110 is adapted to accommodate an object 50 so that the object 50 is stacked on the transparent plate 120 as shown in FIG. 3. The object 50 may be an electronic product such as a smart phone or other types of objects, which is not limited by the disclosure. When the transparent plate 120 supports the object 50, a surface 50a (annotated in FIG. 4) of the object 50 leans against a supporting surface 120a of the transparent plate 120. The ultraviolet light source 130 is adapted to provide ultraviolet light L (shown in FIG. 4). In addition to irradiating an unshielded surface (e.g., a surface 50b) of the object 50, the ultraviolet light L may also pass through an interior of the transparent plate 120 and be transmitted toward the supporting surface 120a of the transparent plate 120, and irradiate the shielded surface 50a of the object 50, achieving comprehensive sterilization.

Furthermore, the sterilization case 100 of this embodiment further includes a reflective layer 140 as shown in FIG. 2 to FIG. 4. The reflective layer 140 is disposed on a bottom surface 112a (annotated in FIG. 4) in the case part 112, and the transparent plate 120 is disposed on the reflective layer 140. The reflective layer 140 may reflect the ultraviolet light L toward the supporting surface 120a of the transparent plate 120.

In this embodiment, a material of the transparent plate 120 may include cyclic block copolymer (CBC), and the transparent plate 120 has good light transmittance for ultraviolet light. In addition, a material of the reflective layer 140 may include Teflon, and the reflective layer 140 has good light reflection efficiency. In other embodiments, the material of the transparent plate 120 may include other light-transmitting materials, and the material of the reflective layer 140 may include other highly reflective materials, which are not limited by the disclosure.

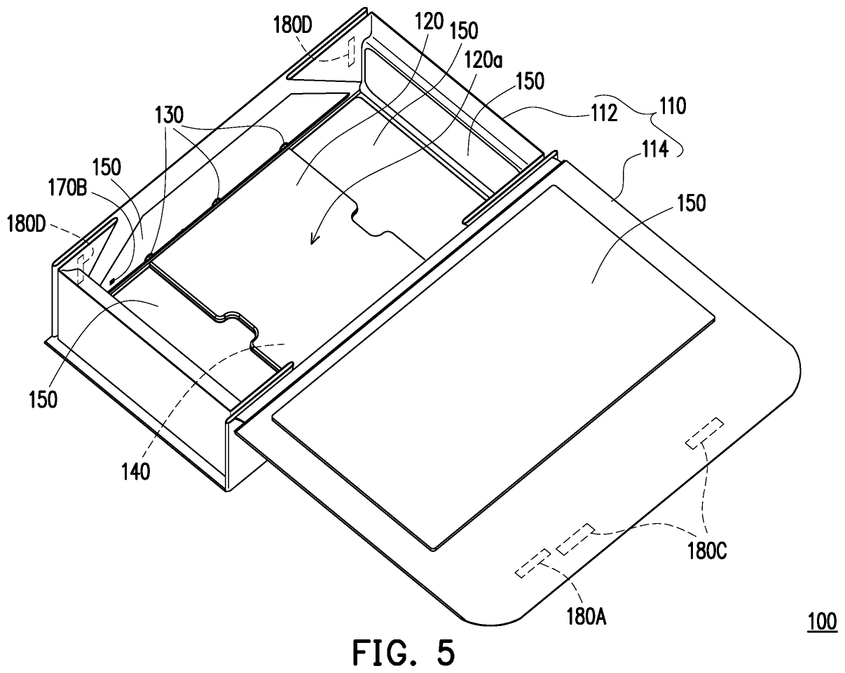

FIG. 5 is a perspective view of the sterilization case of FIG. 3 from another view angle. With reference to FIG. 2 and FIG. 5, the ultraviolet light sources 130 of this embodiment are respectively disposed on two opposite inner surfaces of the case part 112, and a height of the ultraviolet light source 130 relative to the bottom surface 112a in the case part 112 shown in FIG. 2 is different from a height of the ultraviolet light source 130 relative to the bottom surface 112a in the case part 112 shown in FIG. 5. Moreover, the sterilization case 100 further includes a plurality of reflective layers 150. The reflective layers 150 may be metal plates and are respectively disposed on a plurality of inner surfaces of the case part 112, which are adapted to reflect the ultraviolet light L from each ultraviolet light source 130 toward the transparent plate 120 or the object 50. Through disposing the ultraviolet light sources 130 with a height difference as described above, in combination with the reflective layers 150 on the plurality of inner surfaces of the case part 112, the ultraviolet light L irradiates the object 50 (shown in FIG. 3 and FIG. 4) more comprehensively.

The sterilization case 100 of this embodiment includes a status indicator 160 as shown in FIG. 1 and FIG. 2. The status indicator 160 is disposed on an outer side the case body 110. When the ultraviolet light source 130 in the case body 110 is turned on, the status indicator 160 may emit a first color light (e.g., a red light), when the ultraviolet light source 130 in the case body 110 is turned off, the status indicator 160 may emit a second color light (e.g., a green light) different from the first color light, so that a user accordingly knows whether the sterilization case 100 is performing ultraviolet light sterilization. In addition, in this embodiment, the transparent plate 120 may be coated with a photosensitive material such as fluorescent agent. After being irradiated by ultraviolet light, the photosensitive material displays a visible text or graphic that may be maintained for a period of time, so that the user knows that the sterilization case 100 has completed the sterilization.

As shown in FIG. 2, FIG. 3, and FIG. 5, the transparent plate 120 of this embodiment has two finger receiving grooves 120b. The two finger receiving grooves 120b are respectively located on two opposite ends of the supporting surface 120a. Fingers of the user can extend below the object 50 via the finger receiving grooves 120b, which is convenient for the object 50 to be picked up.

With reference to FIG. 2, the sterilization case 100 of this embodiment includes a magnetic sensing element 170A and a magnetic element 180A. The magnetic sensing element 170A is, for example, a magnetic sensor such as a Hall sensor and is disposed in the case part 112. The magnetic element 180A is, for example, a magnet and is disposed in the cover part 114. When the cover part 114 covers the case part 112 as shown in FIG. 1, the magnetic sensing element 170A senses that the magnetic sensing element 170A and the magnetic element 180A are close to each other and accordingly triggers the ultraviolet light source 130 to be turned on. When the cover part 114 is unfolded relative to the case part 112 as shown in FIG. 2, the magnetic sensing element 170A senses that the magnetic sensing element 170A and the magnetic element 180A are away from each other and accordingly triggers the ultraviolet light source 130 to be turned off. Thereby, it is ensured that the ultraviolet light source 130 is only turned on when the cover part 114 covers the case part 112 to prevent the ultraviolet light L from irradiating the user.

In addition, the sterilization case 100 of the embodiment further includes a light sensing element 170B as shown in FIG. 5. The light sensing element 170B is disposed in the case part 112. When the cover part 114 is unfolded relative to the case part 112 as shown in FIG. 2, the light sensing element 170B senses external ambient light and accordingly triggers the ultraviolet light source 130 to be turned off, to further ensure that the ultraviolet light source 130 will not be turned on unexpectedly when the cover part 114 is unfolded relative to the case part 112.

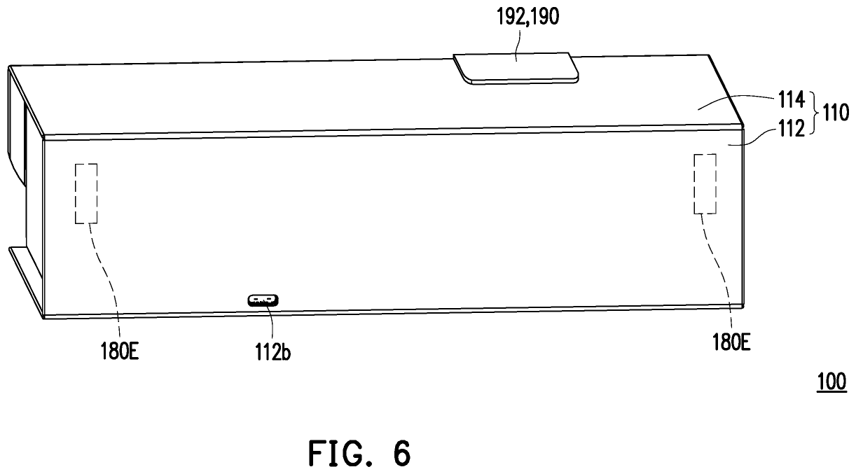

In this embodiment, a wire fixing belt 190 may be added as shown in FIG. 1. The wire fixing belt 190 has a first segment 192 and a second segment 194. The first segment 192 is connected to an outer side of the cover part 114, and the second segment 194 is adapted to be reversely folded relative to the first segment 192, and be attached to the first segment 192 by, for example, magnetic attraction or in other suitable manners as shown in FIG. 1. Thereby, the power cord/charging cord of the sterilization case 100, the power cord/charging cord of the object 50 (shown in FIG. 3 and FIG. 4), or other wires may be fixed between the first segment 192 and the second segment 194. FIG. 6 is a perspective view of the sterilization case of FIG. 1 from another view angle. With reference to FIG. 6, the case body 110 of this embodiment has an electrical slot 112b on the back of the case part 112, where the power cord/charging cord of the sterilization case 100 can be plugged.

With reference to FIG. 2, the sterilization case 100 of this embodiment may further include magnetic elements 180B and magnetic elements 180C. The magnetic elements 180B and the magnetic elements 180C are respectively disposed in the case part 112 and the cover part 114. When the cover part 114 covers the case part 112 as shown in FIG. 1, the cover part 114 is maintained in a covering state through a magnetic attraction force between the magnetic elements 180B and the magnetic elements 180C. The magnetic elements 180B and the magnetic elements 180C may both be magnets, or may respectively be magnets and metal pieces adapted to be magnetically attracted, which are not limited by the disclosure.

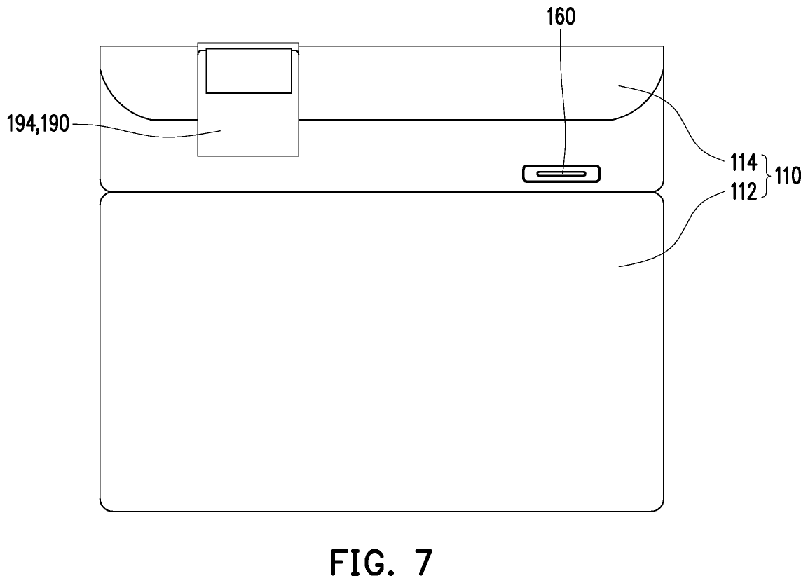

FIG. 7 shows the sterilization case of FIG. 1 folded into a folded state. In this embodiment, the case body 110 of the sterilization case 100 is in a foldable form, which can be expanded into an upright state shown in FIGS. 1 and 2, adapted to accommodate the object 50, and which can be folded into a folded state shown in FIG. 7, convenient for storage and carrying. Furthermore, the sterilization case 100 may further include magnetic elements 180D (shown in FIG. 2 and FIG. 5) and magnetic elements 180E (shown in FIG. 2 and FIG. 6), the magnetic elements 180D and the magnetic elements 180E are disposed in the case part 112 and are in a one-to-one correspondence in the upright state. Thereby, when the case body 110 is in the upright state shown in FIG. 1 and FIG. 2, the case body 110 is maintained in the upright state by magnetic attraction force between each magnetic element 180D and the corresponding magnetic element 180E. The magnetic elements 180D and the magnetic elements 180E may both be magnets, or may respectively be magnets and metal pieces adapted to be magnetically attracted, which are not limited by the disclosure.

On the other hand, the sterilization case 100 may further include magnetic elements 180F (shown in FIG. 2). The magnetic elements 180F are disposed in the case part 112. When the case body 110 is in the folded state shown in FIG. 7, the magnetic elements 180C in the cover part 114 correspond to the magnetic elements 180F in the case part 112. Thereby, the case body 110 is maintained in the folded state by magnetic attraction force between each magnetic element 180C and the corresponding magnetic element 180F. The magnetic elements 180C and the magnetic elements 180F may both be magnets, or may respectively be magnets and metal pieces adapted to be magnetically attracted, which are not limited by the disclosure. In other embodiments, the case body 110 may be folded/unfolded in other suitable manners and maintained in the folded/unfolded state in other appropriate manners, which is not limited by the disclosure.

In summary of the foregoing, in the sterilization case in the disclosure, the transparent plate that supports the object to be sterilized is disposed in the case body, so that the ultraviolet light emitted by the ultraviolet light sources passes through the transparent plate and irradiates the surface of the object that leans against the transparent plate, achieving comprehensive sterilization. Besides, through disposing the ultraviolet light sources in the case body with a height difference, in combination with the reflective layers disposed below the transparent plate and on the plurality of inner surfaces of the case body, the ultraviolet light sources irradiates the object in the case body more comprehensively.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A sterilization case, comprising:
a case body adapted to hold an object;
a transparent plate disposed in the case body and having a supporting surface, wherein when the transparent plate supports the object, a surface of the object leans against the supporting surface; and
at least one ultraviolet light source disposed in the case body and adapted to provide ultraviolet light, wherein the ultraviolet light passes through an interior of the transparent plate and is transmitted toward the supporting surface to irradiate the surface of the object.

2. The sterilization case according to claim 1, wherein a material of the transparent plate comprises cyclic block copolymer.

3. The sterilization case according to claim 1, further comprising a reflective layer, wherein the reflective layer is disposed on a bottom surface of the case body, the transparent plate is disposed on the reflective layer, and the reflective layer is adapted to reflect the ultraviolet light toward the supporting surface.

4. The sterilization case according to claim 3, wherein a material of the reflective layer comprises Teflon.

5. The sterilization case according to claim 1, further comprising at least one reflective layer, wherein the at least one reflective layer is disposed on at least one inner surface of the case body and is adapted to reflect the ultraviolet light toward the transparent plate or the object.

6. The sterilization case according to claim 5, wherein a material of the at least one reflective layer comprises metal.

7. The sterilization case according to claim 1, wherein the at least one ultraviolet light source comprises a plurality of ultraviolet light sources, and a height of a part of the plurality of ultraviolet light sources relative to a bottom surface of the case body is different from a height of another part of the plurality of ultraviolet light sources relative to the bottom surface of the case body.

8. The sterilization case according to claim 1, wherein the transparent plate has a photosensitive material, and the photosensitive material is adapted to display a visible text or graphic after being irradiated by the ultraviolet light.

9. The sterilization case according to claim 1, wherein the case body is adapted to be folded into a folded state and adapted to be unfolded into an upright state.

10. The sterilization case according to claim 1, further comprising a magnetic element and a magnetic sensing element, wherein the case body comprises a case part and a cover part connected to each other, the transparent plate and the at least one ultraviolet light source are disposed in the case part, the magnetic element is disposed in one of the case part and the cover part, and the magnetic sensing element is disposed in the other one of the case part and the cover part, wherein when the cover part covers the case part, the magnetic sensing element senses that the magnetic sensing element and the magnetic element are close to each other and accordingly triggers the at least one ultraviolet light source to be turned on, and when the cover part is unfolded relative to the case part, the magnetic sensing element senses that the magnetic sensing element and the magnetic element are away from each other and accordingly triggers the at least one ultraviolet light source to be turned off.

11. The sterilization case according to claim 1, further comprising a light sensing element, wherein the case body comprises a case part and a cover part connected to each other, and the transparent plate, the at least one ultraviolet light source, and the light sensing element are disposed in the case part, wherein when the cover part is unfolded relative to the case part, the light sensing element senses external ambient light and accordingly triggers the at least one ultraviolet light source to be turned off.

12. The sterilization case according to claim 1, further comprising a wire fixing belt, the wire fixing belt having a first segment and a second segment, wherein the first segment is connected to an outer side of the case body, and the second segment is adapted to be reversely folded relative to the first segment and attached to the first segment.

13. The sterilization case according to claim 1, further comprising a status indicator, the status indicator being disposed on an outer side of the case body, wherein when the at least one ultraviolet light source is turned on, the status indicator emits a first color light, and when the at least one ultraviolet light source is turned off, the status indicator emits a second color light different from the first color light.

14. The sterilization case according to claim 1, wherein the transparent plate has two finger receiving grooves, and the two finger receiving grooves are respectively located on two opposite ends of the supporting surface.
